(19) United States
(12) Patent Application Publication
(10) Pub. No.: US 2019/0307936 A1
(43) Pub. Date: Oct. 10, 2019

(54) Agitator Unit for Use with a Blood Product Storage System and Blood Product Storage System

(71) Applicant: B Medical Systems S.A. R.L., Hosingen (LU)

(72) Inventors: Alan Rushing, Mont-Saint-Guibert (BE); Marc Foyen, Mont-Saint-Guibert (BE)

(73) Assignee: B Medical Systems S.A. R.L., Hosingen (LU)

(21) Appl. No.: 16/315,775

(22) PCT Filed: Jul. 3, 2017
(86) PCT No.: PCT/EP2017/066530

§ 371 Date: Jan. 7, 2019

(30) Foreign Application Priority Data
Jul. 11, 2016 (DE) 10 2016 212 608.3

(51) Int. Cl. A61M 1/02; B01F 11/00; B01F 15/06

(52) U.S. Cl. CPC A61M 1/0254; B01F 11/0014; B01F 2215/0034; A61M 2202/0427; A61M 2205/362; B01F 15/065

(57) ABSTRACT

The present disclosure relates to an agitator unit for use with a blood product storage system, wherein the agitator unit has a movable compartment to receive blood products and a drive for movement of the compartment. The drive of the agitator unit has at least one motor and a gear system with at least one planetary gear unit, wherein the planetary gear unit has at least one planet wheel, which is eccentrically movable by means of the motor, and a planet disc, wherein the planet wheel is connected to the compartment by means of a spigot and rolls on an internal periphery of the planet disc, wherein the spigot is adjustable in its relative position. In addition, the disclosure relates to a modular blood product storage system with an agitator unit.

The present invention relates to an agitator unit for use with a blood product storage system, wherein the agitator unit has a movable compartment to receive blood products and a drive for movement of the compartment. In particular, the invention relates to an agitator unit to receive platelet concentrates disposed in bags. In addition, the present invention relates to a blood product storage system with an agitator unit according to the invention. The latter is also known as a shaker or rocker.

Special storage systems are generally used for storing platelet concentrates that maintain the temperature in the predetermined range and additionally have an agitator unit, which keeps the platelet concentrates in constant, principally rotating movement. These storage systems often also have a monitoring and alarm system to immediately indicate a deviation in temperature or faults of the agitator unit, so that damage of the platelet concentrates can be prevented by quick action.

Therefore, storage systems are known from the prior art, which consist of an incubator cabinet, in which at least one agitator unit is housed as a separate device. The incubator cabinet has a temperature regulation unit to maintain the temperature inside the cabinet in the necessary range of +22±2° C. The agitator unit generally consists of a base plate with a motion generator, by means of which a frame with several tray-type shelves to receive the platelet concentrates disposed in bags is then set in motion.

Although these storage systems can store the platelet concentrates to a high quality, these are nevertheless associated with some disadvantages. For instance, individual adjustment to the actual storage requirement for platelet concentrates is not possible. If the incubator cabinet provides space for four agitator units each with seven compartments, for example, the entire interior of the incubator cabinet must always be regulated in temperature, even if only one compartment of an agitator unit is filled with platelet concentrates disposed in bags. Moreover, in this case the entire agitator unit must also be operated, i.e. so that all seven compartments are kept in motion by means of the common frame.

It is also a disadvantage that all the tray-type compartments of the known agitator units are moved at the same speed. This is particularly disadvantageous when blood products of different ages are housed in the blood product storage system or the agitator unit. In particular, platelet concentrate generally has a life of four days at maximum, wherein the quality decreases with increasing age. The age-related decrease in quality can be counteracted to some extent by an adjustment of the movement. A change in the movement is not possible in the known agitator units. Instead, the compartments of the known agitator units always perform only one movement, e.g. a purely lateral movement.

It is therefore an object of the present invention to provide an improved agitator unit for use in a blood product storage system as well as a blood product storage system with such an agitator unit.

The object is achieved with an agitator unit according to claim 1 and a blood product storage system according to claim 12. Advantageous further embodiments are described in the dependent claims.

The agitator according to the invention is distinguished in particular in that the drive of the agitator unit has at least one motor and a gear system with at least one planetary gear unit, wherein the planetary gear unit has at least one planet wheel, which is eccentrically movable by means of the motor, and a planet disc, wherein the planet wheel is connected to the compartment by means of a spigot and rolls on an internal periphery of the planet disc, wherein the spigot is adjustable in its relative position.

This has the advantage that a single compartment of the agitator unit is moved by means of the gear system. Moreover, the adjustable relative position of the spigot provides the possibility of adapting the type of movement of the compartment by changing the relative position of the spigot. As a result, the movement of the compartment of the agitator unit can be adapted to the necessary given conditions.

It is advantageous if the gear system has precisely two identical planetary gear units. A particular stable and uniform movement of the compartment can thus be obtained.

It is advantageous if the motor drives the gear system by means of a belt. The belt can be a flat belt, a V-belt or a toothed belt, for example. The belt allows the torque of the motor to be transferred to the gear system substantially free from slippage. Moreover, the belt also enables the motor to be positioned remotely from the gear system, since there is no need for a direct shaft connection between the motor and the gear system.

The planet wheel is expediently replaceable, so that the relative position of the spigot on the planet wheel is adjustable by replacing the planet wheel. When the relative position of the spigot on the planet wheel is to be changed, the planet wheel is then removed from the shaft driven via the belt and replaced by another planet wheel with the spigot in a different relative position.

It is expedient in this case if the agitator unit has at least three types of removable planet wheels, wherein the spigot is arranged concentrically on a first planet wheel, so that a rotating movement of the spigot relative to the planet disc results. The spigot is arranged eccentrically and non-tangentially on a second planet wheel, so that an elliptical movement of the spigot relative to the planet disc results. The spigot is arranged tangentially on a third planet wheel, so that a linear movement of the spigot relative to the planet disc results. Of course, the agitator unit can also have only two or more than three replaceable planet wheels, depending on what requirement is set for adaptation of the type of movement of the compartment.

Alternatively, it is advantageous if the spigot is arranged to be movable on the planet wheel between a concentric position with the planet wheel and a tangential position on the planet wheel. The type of movement of the compartment can thus be adjusted quickly and simply by a change of the relative position of the spigot on the planet wheel. If the spigot is in a tangential position, there results a linear movement of the compartment. A shift of the spigot in the direction of the concentric position firstly results in an elliptical movement, which ultimately changes into a circular movement when the spigot is in the concentric position. It is conceivable, for example, that the planet wheel has a multiplicity of spigot seats, in which the spigot can be received. For example, the spigot can be screwed into the spigot seats, so that a quick and easy change of the position of the spigot on the planet wheel can occur by screwing the spigot into another spigot seat.

It is also conceivable that the spigot is movable by means of a moving mechanism. For example, the moving mechanism can be formed by means of a rail system on the planet wheel, wherein the spigot is clamped in the desired position on the rail. A continuous change of the relative position of the spigot on the planet wheel can thus occur, thus enabling a still more selective adjustment of the type of movement of the compartment.

It is advantageous in particular if the moving mechanism has a linear guide that is immovable in relation to the planet wheel and a slotted link guide that is rotatable in relation to the planet wheel, wherein the spigot is guided in the linear guide and the slotted link guide. A forced linear position change of the spigot can thus occur by rotation of the slotted link guide. This allows a particularly fine and simple position adjustment of the spigot. Alternatively, it is conceivable that the slotted link guide is immovable in relation to the planet wheel and the linear guide is rotatable in relation to the planet wheel.

Expediently, the slotted link guide or the linear guide is rotatable in relation to the planet wheel by motor. It is conceivable that the slotted link guide or the linear guide is rotated by means of an electric motor. Thus, the planet wheel does not need to be accessible for the change of position of the spigot. Instead, the rotation of the slotted link guide or the linear guide by motor and thus the position change of the spigot on the planet wheel can also occur by actuation of the electric motor. Moreover, the electric motor can be configured so that its holding power is sufficient to prevent any unwanted rotation of the slotted link guide or the linear guide. It is also conceivable in this regard that the electric motor is supplied with power by means of sliding contacts.

Expediently, the agitator unit has an access opening for the insertion and removal of blood products, wherein the access opening can be closed by a door and wherein the drive stops when the door is opened. This enables a secure and simple removal and insertion of blood products. It is conceivable that the door stops the motor by means of a contact switch. This is particularly advantageous where each agitator unit has its own drive for movement of its own compartment and stoppage of an agitator unit's drive does not interrupt movement of the moveable compartments of other agitator units.

The invention also relates to a modular blood product storage system for temperature-regulated storage of blood products with a temperature regulation unit to regulate the temperature of the interior of the blood product storage system, a base unit and at least one agitator unit according to the invention, as described above. The agitator unit has an upper connection side and a lower connection side, wherein the upper connection side of the agitator unit is selectively connectable to the temperature regulation unit or a further agitator unit, and wherein the lower connection side of the agitator unit is selectively connectable to the base unit or a further agitator unit.

Therefore, in the simplest case the modular blood product storage system consists of a base unit, precisely one agitator unit according to the invention arranged thereon and a temperature regulation unit arranged on the agitator unit. In this case, the temperature regulation unit only has to regulate the temperature of the interior of one agitator unit. Moreover, since the agitator unit has its own drive for movement of the compartment, no unnecessary movement of an empty compartment is performed.

In the case where several blood products are to be stored at regulated temperature, a further agitator unit is arranged between the temperature regulation unit and the base unit. Accordingly, in the case of a decrease in storage requirement one agitator unit or several agitator units can also be removed.

Thus, overall, a modular blood product storage system is provided that can be adjusted quickly and simply to the actually existing storage requirement for blood products. Hence, ultimately only the currently necessary space of the blood product storage system is regulated in temperature by the temperature regulation unit in the desired range of, for example, +22±2° C.

Expediently, the temperature regulation unit and/or the base unit has an air circulation device, wherein the air circulation device generates a temperature-regulated air flow between the temperature regulation unit and the base unit. Thus, the blood product storage system can be regulated effectively to the desired temperature of, for example, +22±2° C. For this it is expedient if the temperature regulation unit has elements for cooling and/or heating, so that the desired temperature can be maintained in the blood product storage system irrespective of the ambient temperature. Alternatively, the elements for cooling and/or heating can also be (additionally) provided in the base unit.

It is advantageous if the agitator unit has a bottom provided with openings, wherein the bottom is arranged below the compartment in the region of the lower connection side and the temperature-regulated air flow flows through the openings from the temperature regulation unit to the base unit. This ensures that all the blood products received in the compartment are regulated to the desired temperature. Moreover, it is thus also ensured that a homogeneous temperature regulation is achieved within the blood product storage system, since the temperature-regulated air flow from the temperature regulation unit can flow through several agitator units. In association with this it can also be advantageous if the compartment has passages for the air flow.

Expediently, the agitator unit has a separate air passage, wherein the air flow from the base unit to the temperature regulation unit flows through the air passage. It can thus be ensured that any mixture of fresh temperature-regulated air and air already changed in temperature does not adversely affect the temperature inside the blood product storage system. As a result, it is guaranteed that the blood products received in the compartment are regulated to the necessary temperature of, preferably, 22±2° C.

It is advantageous if the temperature regulation unit has a power supply, wherein the at least one agitator unit can be supplied with current from the power supply via the upper connection side. In particular, it is advantageous if the power transmission is achieved by means of corresponding contacts or a high-speed coupling. It is advantageous if the agitator unit on the lower connection side also has corresponding elements for power transmission. Thus, a secure power supply can also be guaranteed for a blood product storage system with more than one agitator unit. Alternatively, it is conceivable that the base unit has a power supply, wherein the at least one agitator unit can be supplied via the lower connection side with current from the power supply of the base unit.

As already mentioned above, the agitator unit has an access opening with a door for the insertion and removal of blood products. This can firstly prevent too great an influence on the temperature inside the blood product storage system. Thus, there is no need to open a large-surface door leaf, as in the case of an incubator cabinet, but only a door that closes precisely one compartment off from the surrounding area. The access opening necessary for this is correspondingly small. Moreover, a quick and secure removal or insertion of the blood product can also occur as a result of the stoppage of the drive.

It is advantageous if the blood product storage system has a control device, wherein the drive of the agitator unit can be actuated on an individual basis by means of the control device. It is expedient in this case if the operating parameters of the drive of the agitator unit are adjustable by means of the control device, in particular the speed and type of movement of the compartment. It is thus possible when using several agitator units to adjust the movement of the compartment of each one of the agitator units separately. Thus, for example, the compartment of one agitator unit can move in a circular motion at a speed of 60 rpm, while the compartment of a further agitator unit is moved elliptically at a speed of 50 rpm. This is particularly advantageous when blood products of different ages are housed in the blood product storage system. In particular, platelet concentrate generally has a life of four days at maximum, wherein the quality decreases with increasing age. The age-related decrease in quality can be counteracted to some extent by the adjustment of the movement according to the invention. Expediently, either the temperature regulation unit or the base unit has the control device.

For actuation of the drive of the agitator unit, the blood product storage system expediently has a modular bus system. The bus system can be configured in parallel to the power supply, so that the upper connection side and the lower connection side of the agitator unit are configured to integrate the agitator unit or a further agitator unit into the bus system. A particularly simple and effective actuation of the individual agitator units of the blood product storage system can occur in this way.

In a further embodiment, the agitator unit has a liquid circuit that is connectable to the temperature regulation unit, wherein the temperature regulation unit has a pump for circulation of the fluid of the liquid circuit. As a result of the liquid circuit of each agitator unit it can be guaranteed that even when a plurality of agitator units are used, the temperature in the interior of the blood product storage system remains in the desired range, notably of 22±2° C. It is particularly expedient if the liquid circuit runs in a meander shape or loops below the compartment in order to achieve an effective temperature regulation of the blood products received in the compartment. It is also conceivable that the circulation pump is housed in the base unit.

It is advantageous if the agitator unit has couplings for the liquid circuit on the lower connection side and/or on the upper connection side. The couplings that are particularly suitable are liquid couplings in the form of high-speed or plug-type couplings, so that the blood product storage system can be supplemented or reduced by one agitator unit quickly and simply when required.

The invention will be explained below on the basis of an exemplary embodiment shown in more detail in the schematic drawings, wherein:

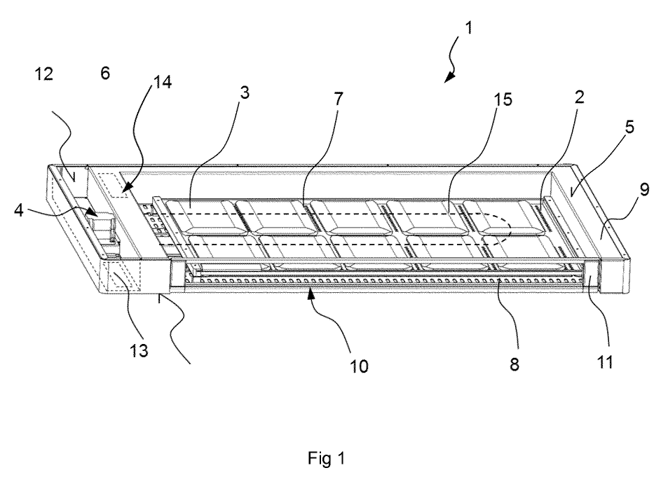

FIG. 1 is a perspective view of an agitator unit according to the invention;

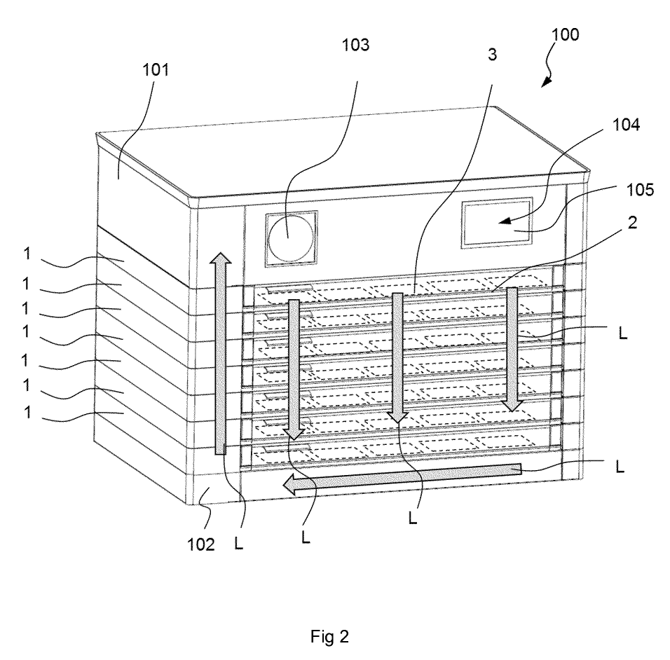

FIG. 2 is a perspective view of a blood product storage system with several agitator units according to the invention;

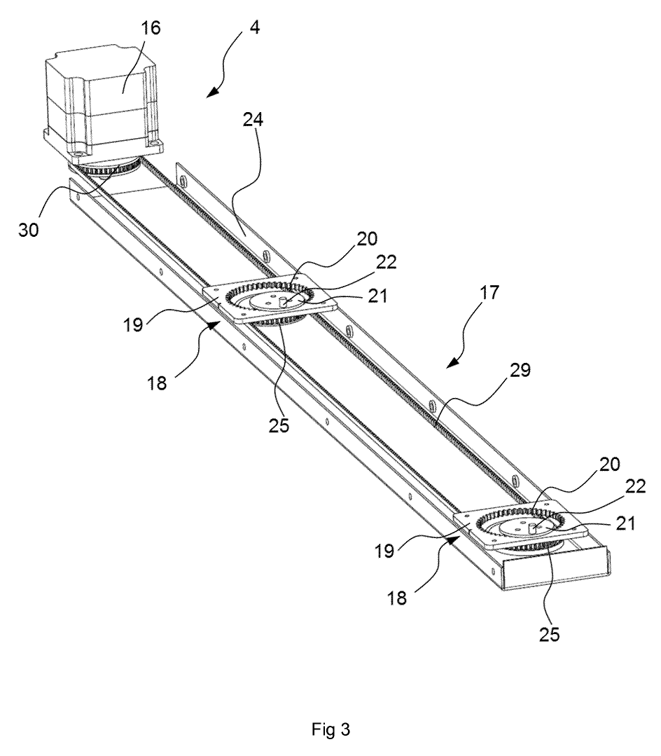

FIG. 3 is a perspective view of the drive of an agitator unit according to the invention;

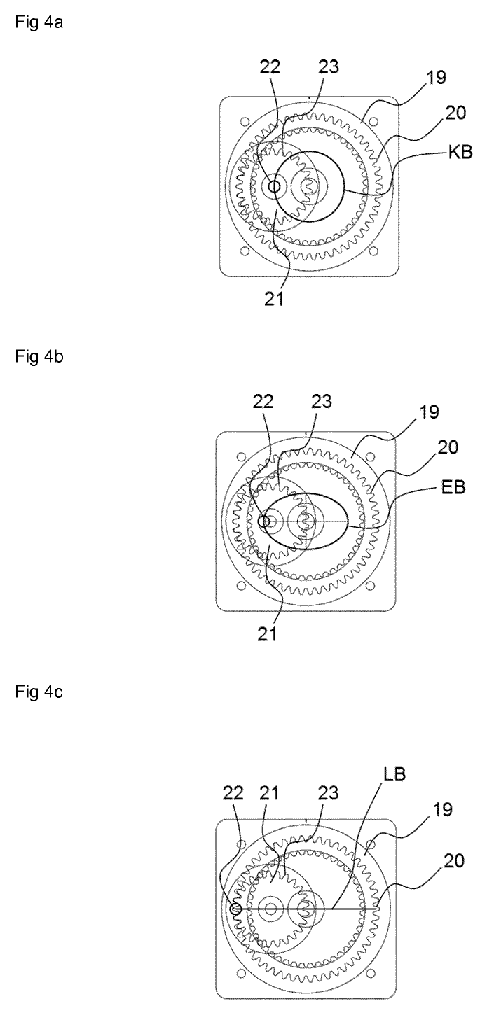

FIG. 4 is a representation of the different positions of the spigot on the planet wheel; and

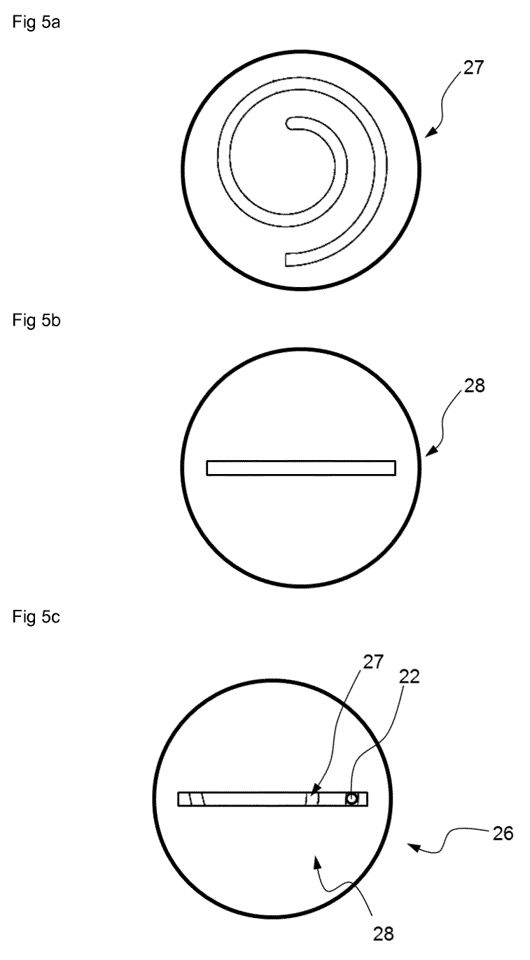

FIG. 5 shows a moving mechanism for movement of the spigot.

FIG. 1 shows an agitator unit 1 according to the invention for use with a blood product storage system 100 (see FIG. 2). The agitator unit 1 has a movable compartment 2 to receive blood products 3. In this exemplary embodiment platelet concentrate in bags is provided as blood products 3. The compartment 2 is provided with passages 7, as shown. In addition, the agitator unit 1 has a drive 4 for moving the compartment 2. In addition, each agitator unit 1 has an upper connection side 5 and a lower connection side 6.

In addition, the agitator unit 1 has a bottom 8 provided with holes, which is arranged below the compartment 2 in the region of the lower connection side 7. The compartment 2 is surrounded in a U-shape by an insulation 9 so that an access opening 10 is left open for the insertion and removal of blood products 3 into the compartment 2 or from the compartment 2. The access opening 10 can be closed with a door 11. Moreover, the agitator unit 1 has a side chamber 12, in which parts of the drive 9 and also a separate air passage 13 (shown in dotted lines) are arranged. The side chamber 12 is separated from the region, in which the compartment 2 is arranged, by the insulation 9.

The agitator unit 1 has a connection region 14 on the upper connection side 5 and on the lower connection side 6. The connection region 14 serves to connect the agitator unit 1 to a further agitator unit 1 or other components 101, 102 of the blood product storage system 100, as will be explained in more detail below. The connection region 14 has a power coupling, a bus coupling as well as a fluid coupling for connection of a liquid circuit 15 of the agitator unit 1. The liquid circuit 15 of the agitator unit 1 is directed in a loop or meandering shape below the compartment 2 and serves to (additionally) regulate the temperature of the blood products 3 received in the compartment 2, as will be explained further below in association with the blood product storage system 100 with reference to FIG. 2.

The drive 4 of the agitator unit 1 will be described in the following. The drive 4 has an electric motor 16 and a gear system 17. The gear system 17 has two planetary gear units 18. The planetary gear units 18 are identical in structure, so that only one planetary gear unit 18 will be described in more detail below.

The planetary gear unit 18 consists of a planet disc 19 with a toothed internal periphery 20. A planet wheel 21 with a spigot 22 and a toothed external periphery 23 can rotate on the toothed internal periphery 20. The planet disc 19 is fixedly connected to a frame 24 of the drive 4. The planet wheel 21 is driven eccentrically by means of a pulley 25. The pulley 25 is arranged concentrically to the planet disc 19 and connected by means of a belt 29 to a drive wheel 30 of the electric motor 16. The belt 29 is configured as a toothed belt here, which meshes with the two shown pulleys 25 and the drive wheel 30.

As shown, the spigot 22 is arranged eccentrically on the planet wheel 21 and extends in vertical direction from the frame 24. The spigot 22 is connected to the compartment 2 of the agitator unit 1 and transfers the resulting movement to the compartment 2. An elliptical movement of the compartment 8 is achieved as a result of the eccentric arrangement of the planet wheel 21 to the pulley 25 and the eccentric arrangement of the spigot 22 on the planet wheel 21.

The type of movement of the compartment 2 can be influenced by changing the relative position of the spigot 22 on the planet wheel 21. As mentioned, a rotating movement EB of the compartment 2 results in the case of the eccentric position of the spigot 22 on the planet wheel 21 shown in FIG. 3 and FIG. 4b. A rotating movement EB of the compartment 2 results in the case of the concentric arrangement of the spigot 22 on the planet wheel 21 shown in FIG. 4a. A linear movement LB of the compartment 2 results in the case of the tangential arrangement of the spigot 22 on the planet wheel 21 shown in FIG. 4c, i.e. an arrangement substantially in the region of the toothed external periphery 23.

The desired position of the spigot 22 on the planet wheel 21 can firstly be achieved by several replaceable planet wheels 21 being provided, on which the spigot 22 is respectively arranged in another relative position. For example, three types of planet wheel 21 can thus be provided, which have the spigot 22 at the relative positions shown in FIGS. 4a to 4c. Thus, the type of movement of the compartment 2 can be adjusted by replacing the planet wheel 21.

In addition, it is also possible to change the relative position of the spigot 22 on the planet wheel 21 by a moving mechanism 26, see FIG. 5c. In this case, only one planet wheel 21 with a spigot 22 movably arranged thereon is provided. The moving mechanism 26 has a slotted link guide 27 and a linear guide 28. In the shown exemplary embodiment the slotted link guide 27 is a worm guide. The spigot 22 is guided in both guides 27, 28, as is shown in a simplified manner in FIG. 5c. One of the two guides 27, 28 is immovable in relation to the planet wheel 21, while the other of the two guides 27, 28 is rotatable in relation to the planet wheel 21. For example, the slotted link guide 27 can be rotatably mounted. If the slotted link guide 27 is now rotated with the spigot 22 guided therein, this slides along the slotted link guide 27. As a result of the additional forced guidance of the spigot 22 in the linear guide 28, there thus results a necessarily linear movement of the spigot 22 along the linear guide 28. The rotation of the slotted link guide 28 can occur by means of an electric motor, for example, which is supplied with power by means of sliding contacts.

FIG. 2 shows a blood product storage system 100 according to the invention for the temperature-regulated storage of blood products 2 with a temperature regulation unit 101, a base unit 102 and, in this exemplary embodiment, seven agitator units 1. The blood product storage system 100 has a layered vertical structure, wherein the base unit 102 forms the bottom unit and the temperature regulation unit 101 the top unit in vertical direction. Moreover, the base unit 102 is conceived to stand the blood product storage system 100 upright, e.g. on a table or the floor. For this, the base unit 102 can have rotating feet (not shown) on the underside to also place the blood product storage system 1 horizontally on uneven support surfaces.

The temperature regulation unit 101 and/or the base unit 102 has an air circulation device (not shown) for generation of a temperature-regulated air flow L. The temperature-regulated air flow L is represented by arrows in FIG. 2. In this exemplary embodiment the temperature-regulated air flow L is generated by the temperature regulation unit 101. The temperature-regulated air flow L regulates the interior of the blood product storage system 100 to the temperature necessary for platelet concentrates of 22±2° C. by flowing from the temperature regulation unit 101 through the agitator units 1 to the base unit 102. In the base unit 102 the air flow L is then directed laterally in the direction of the side chamber 12 of the agitator units 1, and through the separate air passages 13 of the agitator units 1 from the base unit 102 again in the direction of the temperature regulation unit 101. It can thus be ensured that the blood products 3 are regulated to the desired temperature with freshly temperature-regulated air. To enable or not hinder the air flow L, each agitator unit 1 has passages 7 in the respective compartment 2 as well as the bottom 8 provided with openings.

Moreover, the (additional) liquid circuit 15 of each of the agitator units 1 is provided for temperature regulation of the blood products 3. In particular if the blood product storage system 100 has a relatively high number of agitator units 1 (e.g. 24), regulation to the desired temperature can be ensured by the additional liquid circuit 15. For this, a combined liquid circuit is generated by means of the fluid couplings provided in the connection region 14, into which all the individual liquid circuits 15 of the respective agitator units 1 are incorporated. The temperature regulation unit 101 or the base unit 102 has a pump for circulating the combined liquid circuit. The combined liquid circuit is preferably pre-regulated to the desired temperature by means of an additional temperature regulation element.

The temperature regulation unit 101 additionally has a power supply (not shown), a temperature curve display 103, a control device 104 and also a control panel 105. The control panel 105 is configured as a touch screen in this exemplary embodiment. The temperature curve display 103 shows the current temperature in the interior of the blood product storage system 100 as well as a temperature-time curve. The control device 104 controls all functions of the temperature regulation unit 101 and the incorporated agitator units 1. The power supply in this exemplary embodiment is a conventional 220 V connection, but can also have additional batteries, for example, to also guarantee function of the blood product storage system 100 in the event of a power cut. Moreover, the power supply can also be arranged in the base unit 102.

For construction of the blood product storage system 100 the base unit 102 is firstly erected. The first agitator 1 is placed with the lower connection side 6 on the base unit 102 and is firmly connected to this, for example, by means of one or more connecting elements. A second agitator unit 1 can then be placed with the lower connection side 6 on the upper connection side 5 of this first agitator unit 1. In this case, the liquid circuit 15 of the first agitator unit 1 is connected to the liquid circuit 15 of the second agitator unit 1 by means of the fluid couplings provided in connection region 14. In addition, a power-carrying connection is created between the first agitator unit 1 and the second agitator unit 1 by means of the contacts provided in the respective connection region 15. A bus connection is also created accordingly by means of a contact or a coupling. A further agitator unit 1 can then be placed with the lower connection side 6 onto the upper connection side 5 of the second agitator unit 1 and integrated as just described. The temperature regulation unit 101 is lastly placed onto the upper connection side 6 of the last desired agitator unit 1. In this case, the combined liquid circuit is connected to the circulation pump of the temperature regulation unit 101 by means of corresponding fluid couplings. In addition, the power supply of the agitator units 1 is created by a connection with the power supply of the temperature regulation unit 101. Moreover, the agitator units 1 are integrated into a bus system connected to the control device 104.

The entire blood product storage system 100 can now be controlled by means of the control device 104 via control panel 105. For example, the combined liquid circuit can be activated or deactivated. In addition, the drive 4 of an agitator unit 1 can be actuated selectively, for example, and thus be reduced in speed, for example. In the case where the agitator units 1 have a motor-driven moving mechanism 26, it is also possible to adjust the relative position of the spigot 22 on the planet wheel, and thus also the resulting movement of the compartment 2, via the control panel 105. In addition, the control panel 105 can also be used to cause the drive 4 of an agitator unit 1 to stop when the door 11 of the access opening 10 is opened. It is also conceivable that the drive 4 is connected to the door 11 by means of a contact switch, so that stoppage of the drive 4 can occur independently of an actuation through the control device 104.

For the modular extension and reduction of the storage capacity of the blood product storage system 100 the temperature regulation unit 101 is firstly removed from the upper connection side 5 of the uppermost agitator unit 1. Either additional agitator units 1 can then be put in place or agitator units 1 can be removed. Once the desired storage capacity has been reached, the temperature regulation unit 101 is placed on the upper connection side 5 of the now uppermost agitator unit 1 again.

### LIST OF REFERENCES

1 agitator unit

2 compartment

3 blood product/platelet concentrate

4 drive

5 upper connection side

6 lower connection side

7 passage

8 bottom

9 insulation

10 access opening

11 door

12 side chamber

13 air passage

14 connection region

15 liquid circuit

16 motor

17 gear system

18 gear unit

19 planet disc

20 toothed internal periphery

21 planet wheel

22 spigot

23 toothed external periphery

24 frame

25 pulley

26 moving mechanism

27 slotted link guide

28 linear guide

29 belt

30 drive wheel

100 blood product storage system

101 temperature regulation unit

102 base unit

103 temperature display

104 control device

105 control panel

L air flow

EB elliptical movement

KB rotating movement

LB linear movement

1.-14. (canceled)

15. Agitator unit for use with a blood product storage system, wherein the agitator unit has a movable compartment to receive blood products and a drive for movement of the compartment, wherein the drive of the agitator unit has at least one motor and a gear system with at least one planetary gear unit, wherein the planetary gear unit has at least one planet wheel, which is eccentrically movable by means of the motor, and a planet disc, wherein the planet wheel is connected to the compartment by means of a spigot and rolls on an internal periphery of the planet disc, and wherein the spigot is adjustable in its relative position.

16. The agitator unit of claim 15, wherein the gear system has precisely two identical planetary gear units.

17. The agitator unit of claim 15, wherein the motor drives the gear system by means of a belt.

18. The agitator unit of claim 15, wherein the planet wheel is replaceable, so that the relative position of the spigot on the planet wheel is adjustable by replacing the planet wheel.

19. The agitator unit of claim 18, wherein the agitator unit has at least three removable planet wheels, wherein the spigot is arranged concentrically on a first planet wheel, so that a rotating movement of the spigot relative to the planet disc results, wherein the spigot is arranged eccentrically and non-tangentially on a second planet wheel, so that an elliptical movement of the spigot relative to the planet disc results; and wherein the spigot is arranged tangentially on a third planet wheel, so that a linear movement of the spigot relative to the planet disc results.

20. The agitator unit of claim 15, wherein the spigot (is arranged to be movable on the planet wheel, notably between a concentric position with the planet wheel and a tangential position on the planet wheel.

21. The agitator unit of claim 20, wherein the spigot is movable by means of a moving mechanism.

22. The agitator unit of claim 21, wherein the moving mechanism has a linear guide that is immovable in relation to the planet wheel and a slotted link guide that is rotatable in relation to the planet wheel, wherein the spigot is guided in the linear guide and the slotted link guide.

23. The agitator unit of claim 21, wherein the moving mechanism has a linear guide that is rotatable in relation to the planet wheel and a slotted link guide that is immovable in relation to the planet wheel, wherein the spigot is guided in the linear guide and the slotted link guide.

24. The agitator unit of claim 15, wherein the agitator unit has a guide selected from a slotted link guide and a linear guide which is rotatable in relation to the planet wheel by a motor.

25. The agitator unit of claim 15, wherein the agitator unit has an access opening for the insertion and removal of blood products, wherein the access opening can be closed by a door and wherein the drive stops when the door is opened.

26. Modular blood product storage system for temperature-regulated storage of blood products comprising a temperature regulation unit to regulate the temperature of the interior of the blood product storage system, a base unit and at least one agitator unit according to claim 15, wherein the agitator unit has an upper connection side and a lower connection side, wherein the upper connection side of the agitator unit is selectively connectable to the temperature regulation unit or a further agitator unit, and wherein the lower connection side of the agitator unit is selectively connectable to a unit selected from the base unit and a further agitator unit.

27. The modular blood product storage system of claim 26, wherein the modular blood storage system comprises a plurality of agitator units and in which each agitator unit has its own drive for movement of its compartment.

28. The modular blood product storage system of claim 27, wherein each agitator unit has an access opening for the insertion and removal of blood products, wherein the access opening can be closed by a door and wherein the drive of the agitator unit stops when the door of the agitator unit is opened.

29. Agitator unit
wherein the agitator unit is an agitator unit of a blood platelet storage system configured for temperature-regulated storage of blood platelets disposed in bags at a temperature of about +22° C.±2° C.,
wherein the agitator unit has a movable compartment to receive the blood platelets disposed in bags and a drive for movement of the compartment,
wherein the drive of the agitator unit has at least one motor and a gear system with at least one planetary gear unit,
wherein the planetary gear unit has at least one planet wheel, which is eccentrically movable by means of the motor, and a planet disc,
wherein the planet wheel is connected to the compartment by means of a spigot and rolls on an internal periphery of the planet disc, and
wherein the spigot is adjustable in its relative position.
